(19) United States
(12) Patent Application Publication
(10) Pub. No.: US 2015/0297100 A1
(43) Pub. Date: Oct. 22, 2015

(54) Vital Socks

(71) Applicant: HILARIO CASTILLO, (US)

(72) Inventor: HILARIO CASTILLO, Yonkers, NY (US)

(21) Appl. No.: 14/690,379

(22) Filed: Apr. 18, 2015

Related U.S. Application Data
(60) Provisional application No. 61/981,578, filed on Apr. 18, 2014.

(51) Int. Cl. A61B 5/0205; A61B 5/00

(52) U.S. Cl. CPC A61B 5/02055; A61B 5/6802; A61B 5/742; A61B 5/0002; A61B 5/021

(57) ABSTRACT

A wearable apparatus for measuring physiological activities of a user is provided. The wearable apparatus includes a housing worn by the user having variable compression forces and a ring control center attached to the housing. The ring control center includes one or more sensors for measuring the physiological activities of the user, one or more compression control buttons for controlling the compression force of the housing on the user; and a display unit for displaying the value of the measured physiological activities received from the one or more sensors.

### CROSS REFRENCE TO RELATED PATENT APPLICATION

The present invention claims priority of the provisional patent application No. 61981578 filed on Apr. 18, 2014; all of which are incorporated herein by reference in their entireties.

### BACKGROUND OF THE INVENTION

1. Field of the Invention

The present invention generally relates to an apparatus for measuring biological parameters of a user, and more particularly relates to the apparatus for sensing the physiology of the body and controlling the compression level of the user. The apparatus is worn by the user.

2. Description of Related Art

Fabrics in the art are known with intelligence capabilities. Such fabrics have embedded conductive elements to monitor vital signs of the body of the user. Typical methods include interweaving of the conductive elements into the fabric. Such fabrics for obtaining the physiological data should be in contact with the skin of the user.

Various socks are used for measuring the pressure distribution around the foot of diabetic patients. The metallic fibers are connected to an electronic circuit which computes the pressure distribution from the electrical resistances at the intersection between the metallic fibers. However, these socks do not measure the physiological data precisely and lacks display unit for displaying the physiological data measured from the user.

It has been found that the pressure sensors known in the art do not allow precise measurement of a sensed pressure over a sufficiently wide range due to lack of contraction forces. Such wearable devices lack buttons to control compression force. Further, it has been found that the fabric sensing devices in the art are of fixed size so it became difficult for bulky user to use that device. Devices in the art are heavy and big in size so it is difficult to carry those devices.

Therefore there is a need of an apparatus wearable by the user for sensing the physiological parameters of user and monitor heart rate, pulse rate, blood pressure data, body temperature, breathing rate. Further, the apparatus should display the sensed physiological parameters and furthermore contain at least one button to control the contraction forces.

### SUMMARY OF THE INVENTION

In accordance with the teachings of the present invention, wearable apparatus for measuring physiological activities of a user is provided.

An object of the present invention is to provide a wearable apparatus for measuring physiological activities of the user. The wearable apparatus includes a housing worn by the user having variable compression forces and a ring control center attached to the housing for measuring the physiological data of the user.

The ring control center includes one or more sensors for measuring the physiological activities of the user, one or more compression control buttons for controlling the compression force of the housing on the user, a display unit for displaying the value of the measured physiological activities received from the one or more sensors.

Another object of the present invention is to provide the ring control center with the blood pressure button for measuring the blood pressure of the user through the one or more sensors. In yet another object of the present invention is to provide a communication unit for sharing the vital information received from the one or more sensors with an external device. The communication unit further receives signal from the external device to operate at least one of the one or more sensors; one or more compression control buttons; the blood pressure button; and the display unit.

In yet another object of the present invention the ring control center further includes an electronic assembly for embedding at least one of the one or more sensors, the display unit, and the one or more compression control buttons.

Another object of the present invention is to provide the ring control center with a power source for powering the ring control center and the housing. In yet another object of the present invention, the electronic assembly distributes the power received from the power source with the housing and the ring control center. In yet another object of the present invention is to provide a power unit embedded in the electronic assembly for receiving power from an external source.

Further features and advantages of the present invention, as well as the structure and operation of various embodiments of the present invention, are described in detail below with reference to the accompanying drawings.

### BRIEF DESCRIPTION OF DRAWINGS

FIG. 1 illustrates a perspective view of a wearable apparatus for measuring physiological activities of a user in accordance with a preferred embodiment of the present invention; and

FIG. 2 illustrates another perspective view of a wearable apparatus showing a sensor in accordance with another preferred embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF DRAWINGS

While this technology is illustrated and described in a preferred embodiment, a wearable apparatus for measuring physiological activities of a user may be produced in many different configurations, forms, and materials. There is depicted in the drawings, and will herein be described in detail, as a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the technology described herein.

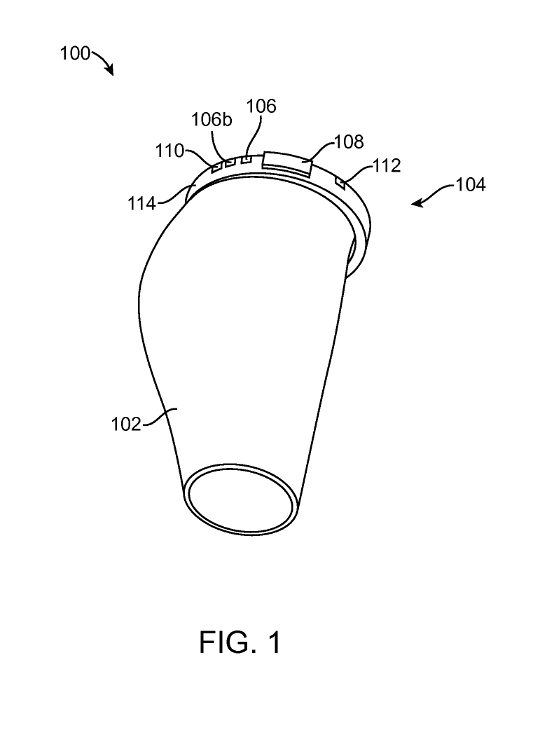

FIG. 1 illustrates a perspective view of a wearable apparatus 100 for measuring physiological activities of a user. The wearable apparatus 100 includes a housing 102 worn by the user having compression forces, and a ring control center 104 attached to the housing 102 for measuring the physiological data of the user. The ring control center 104 includes at least one sensor (not shown in FIG. 1) for measuring the physiological activities of the user, one or more compression control buttons 106 such as 106a and 106b for controlling the contraction forces of the housing 102, and a display unit 108 for displaying the value of the measured physiological activities received from the one or more sensors (not shown in FIG. 1).

Examples of physiological activities includes but not limited to oxygen level, blood flow rate at the popliteal vessels, leg or arm diameter, heart rate compression force, leg temperature, patient heart rate, cardiac rhythm, blood pressure, detect deep vein thrombosis, blood clot and therapeutic regime overtime.

Further, the information may relates to Cardiac Output-volume of blood pumped by heart in one minute (SVXHR) 5-7 L, and Stroke Volume (SV) the volume of blood pumped from one ventricle of the heart with each beat. The electrically conductive material is a silver yarn or a yarn coated with silver. Various commercially available silver yarns are useful for creating the housing 102 of the present invention.

The housing 102 is made up of a material that allows variable compression force. In a preferred embodiment, the housing 102 is made up of silver yarn. The silver yarn comprises 99.9% pure elemental silver and is highly electrically conductive, lightweight, flexible, stretchable, washable, and durable.

In addition, the silver yarn may be of a broad spectrum antimicrobial and odor eliminator useful in the care of wounds such as dermal ulcers. In another preferred embodiment of the present invention, the housing 102 may be made of conductive material such as a copper wire knit and blended with materials such as nylon, cotton, spandex, and natural rubber.

Further, the housing 102 provides greater comfort for the user. In a preferred embodiment, the shape of the housing is of a sock. However, it would be readily apparent to those skilled in the art that various shapes of the housing 102 may also be envisioned such as for gloves, wrist band etc without deviating from the scope of the present invention.

The housing 102 may further be made in various shape and sizes to cater all types of leg, arm and wrist. Further, the material of the housing 102 enables specific monitored compression treatment to different conditions and different swelling levels. In another preferred embodiment of the present invention, the housing 102 may further be worn upon the user's existing sock. The housing 102 is further easily removable and cleaned.

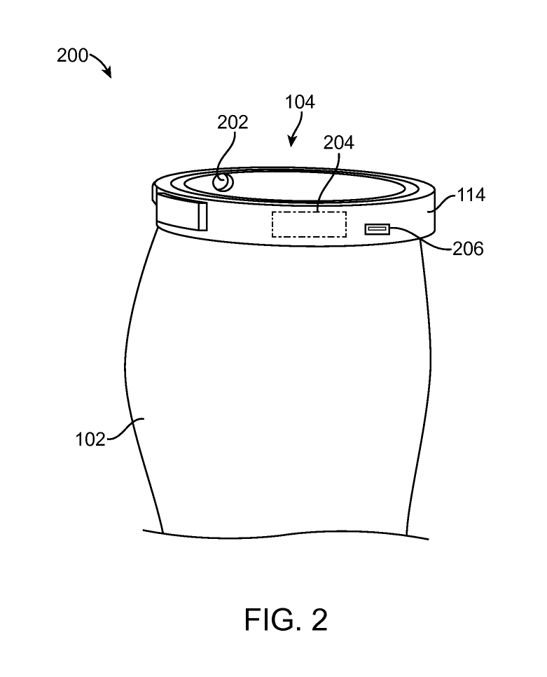

The sensor (not shown in FIG. 1) is explained in detail in conjunction with FIG. 2 of the present invention. The one or more compression control buttons 106 controls the compression forces of the housing 102. In a preferred embodiment of the present invention, the compression control button 106a increases the compression force on the user and the compression control button 106b decreases the compression force on the user.

The display unit 108 displays the signal from the sensor (shown in FIG. 2). Examples of display unit 108 includes but not limited to LED, LCD, Plasma and other similar devices. In another preferred embodiment of the present invention, the display unit 108 has a touch screen to receive commands to operate the sensors (not shown in FIG. 1) and one or more compression control buttons 106.

In another embodiment of the present invention, the ring control center 104 includes a blood pressure button 110 for measuring the blood pressure of the user through the one or more sensors (not shown in FIG. 1). The blood pressure button 110 initiates the sensor (not shown in FIG. 1) to monitor the blood pressure of the user and further displays the blood pressure on the display unit 108. The blood pressure button 110 is a normal on and off button to control the sensor (not shown in FIG. 1) to measure the blood pressure.

In another preferred embodiment of the present invention, the ring control center 104 further comprising a communication unit 112 for sharing the vital information received from the sensor (not shown in FIG. 1) with an external device. Further, the communication unit 112 receives signal from the external device to operate at least one of the one or more sensors (not shown in FIG. 1); one or more compression control buttons 106; the blood pressure button 110; and the display unit 108.

Examples of the communication unit 112 include but not limited to bluetooth, modem, and a wireless unit. Examples of the external device include but not limited to mobile phone, smartphones, tethered device (computers) and other devices with GPS capability. It would be readily apparent to those skilled in the art that various types of the communication unit 112 and external devices may be used without deviating from the scope of the present invention.

For example, in case of a cardiac rhydim chance like a systole (sudden cardiac death) the external device locates the user wearing GPS enabled wearable apparatus 100 and then external device may be programmed

In another preferred embodiment of the present invention, the communication unit 112 is used for sending alerts to the external devices. Examples of alert include alerts for abnormal vital sign readings or parameters outside the user's baseline including sudden cardiac death (asystole). However, it would be readily apparent to those skilled in the art that various other alerts may be send without deviating from the scope of the present invention.

In another preferred embodiment of the present invention, the ring control center 104 further includes an electronic assembly 114 for embedding at least one the one or more sensors, one or more compression control buttons 106, the blood pressure button 110, a display unit 108 and the communication unit 112. Further, the electronic assembly 114 distributes power with the housing 102 and the ring control center 104.

FIG. 2 illustrates another perspective view of the wearable apparatus 200 showing the sensor 202. The sensor 202 senses the physiological activities of the user. In a preferred embodiment of the present invention the sensor 202 is electro-optical. However, it would be readily apparent to those skilled in the art that various types of sensors 202 may also be used for measuring physiological activities without deviating from the scope of the present invention.

In another preferred embodiment of the present invention, the wearable apparatus 200 further includes a power source 204 for powering the ring control center 104 and the housing 102. In a preferred embodiment of the present invention, the power source 204 is a battery. The power source 204 is a small size battery embedded in the electronic assembly 114.

In another preferred embodiment of the present invention, the ring control center 104 further includes a power unit 204 embedded in the electronic circuitry 114 for receiving power from an external source. Examples of the power unit 204 include a USB port, electrical socket etc for receiving power from an external source. The external source may be AC power or any electronic devices that provides power to power the power source 204.

The present invention offers various advantages such as stimulating blood circulation, improves oxygen delivery to the muscles, reduces lactic acid production, provides relief to the cramp, alleviating swelling, and contributing to a boost in performance and well-being.

There has thus been shown and described a wearable apparatus which fulfills all the objects and advantages sought therefore. Many changes, modifications, variations and other uses and applications of the subject invention will, however, become apparent to those skilled in the art after considering this specification and the accompanying drawings, which disclose the preferred embodiments thereof. All such changes, modifications, variations and other uses and applications which do not depart from the spirit and scope of the invention are deemed to be covered by the invention, which is to be limited only by the claims which follow.

1. A wearable apparatus for measuring physiological activities of a user, said wearable apparatus comprising:
a housing worn by the user having variable compression forces; and
a ring control center attached to the housing comprising:
one or more sensors for measuring the physiological activities of the user;
one or more compression control buttons for controlling the compression force of the housing on the user; and
a display unit for displaying the value of the measured physiological activities received from the one or more sensors.

2. The ring control center according to claim 1 further comprising a blood pressure button for measuring the blood pressure of the user through the one or more sensors.

3. The ring control center according to claim 1 further comprising a communication unit for sharing the vital information received from the one or more sensors with an external device.

4. The ring control center according to claim 3 wherein said communication unit further receives signal from the external device to operate at least one of the one or more sensors; one or more compression control buttons; the blood pressure button; and the display unit.

5. The ring control center according to claim 1 further comprising an electronic assembly for embedding at least one of the one or more sensors; the display unit; and the one or more compression control buttons.

6. The wearable apparatus according to claim 1 further comprising a power source for powering the ring control center and the housing.

7. The ring control center according to claim 6 wherein the electronic assembly distributes the power received from the power source with the housing and the ring control center.

8. The ring control center according to claim 5 further comprising a power unit embedded in the electronic assembly for receiving power from an external source.
